(19) United States
(12) Patent Application Publication
(10) Pub. No.: US 2025/0288568 A1
(43) Pub. Date: Sep. 18, 2025

(54) NEW CRYSTAL FORM OF A PDE4 INHIBITOR

(71) Applicant: CHIESI FARMACEUTICI S.P.A., Parma (IT)

(72) Inventors: Francesco AMADEI, Parma (IT); Irene BASSANETTI, Parma (IT); Luca FORNASARI, Parma (IT)

(73) Assignee: CHIESI FARMACEUTICI S.P.A., Parma (IT)

(21) Appl. No.: 18/859,618

(22) PCT Filed: Apr. 26, 2023
(86) PCT No.: PCT/EP2023/060886

§ 371 Date: Oct. 24, 2024

(30) Foreign Application Priority Data
Apr. 27, 2022 (EP) 22170229.3

(51) Int. Cl. A61K 31/44; A61K 9/00; A61P 11/00; C07D 213/89

(52) U.S. Cl. CPC A61K 31/44; A61K 9/0075; A61P 11/00; C07D 213/89

(57) ABSTRACT

The present invention relates to the crystal Form 2 of the compound of formula (I), to the process for its isolation and to the pharmaceutical compositions thereof. The present invention also relates to the crystal Form 2 of the compound of formula (I) for use as a medicament and for the manufacture of a medicament for the prevention and/or treatment of an inflammatory respiratory or obstructive respiratory disease.

### FIELD OF THE INVENTION

The present invention relates to the crystal Form 2 of the compound of formula (I), to the process for its isolation and to the pharmaceutical compositions thereof. The present invention also relates to the crystal Form 2 of the compound of formula (I) for use as a medicament and for the manufacture of a medicament for the prevention and/or treatment of an inflammatory respiratory or obstructive respiratory disease.

### BACKGROUND OF THE INVENTION

The compound of formula (I)

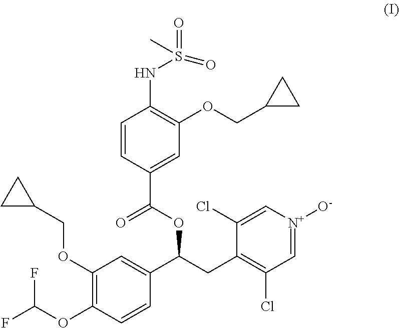 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

also named tanimilast or CHF6001 or CHF-6001, with INN (3,5-dichloro-4-[(2S)-2-[3-(cyclopropylmethoxy)-4-(difluoromethoxy)phenyl]-2-{[3-(cyclopropylmethoxy)-4-(methanesulfonamido)benzoyl]oxy}ethyl]pyridine1-oxide), is an highly potent and selective PDE4 inhibitor with robust anti-inflammatory activity, currently under clinical development.

Compound of formula (I) has been disclosed in prior art documents in the name of Chiesi: WO 2009/018909 directed to its general formula, methods of preparation, compositions and therapeutic use; WO 2010/089107 specifically directed to sulphonamido derivatives as (−) enantiomers, including compound of formula (I), methods of preparation, compositions and therapeutic use; WO 2012/016889 directed to dry powder formulations comprising the compound of formula (I); WO 2015/059050 directed to crystal form, named Form A, of the compound of formula (I) characterized by specific XRPD peaks and the process for obtaining it.

Despite the above cited prior art, there is the need for developing a form of the compound of formula (I) with improved solubility and bioavailability.

In this direction, inventors have surprisingly found that the crystal Form 2 of the compound of formula (I), according to the present invention, has advantageous properties selected from solubility, dissolution rate, low risk of lung accumulation after repeated administration, bioavailability and efficacy.

### SUMMARY OF THE INVENTION

In a first aspect, the present invention refers to the crystal form of a compound of formula (I)

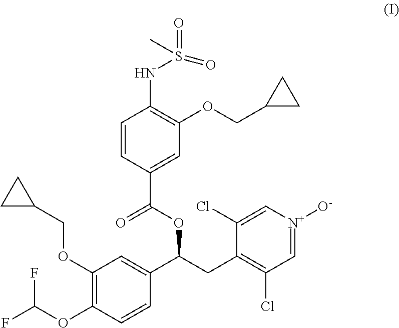 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

designated as crystal Form 2, characterized by at least the following XRPD peaks: 4.0, 7.9, 13.2±0.2 degrees/2 theta [Cu Kα radiation (λ=1.5406 Å)].

In a second aspect, the invention provides a process for the preparation of the crystal Form 2 of the compound of formula (I), characterized by at least the following XRPD peaks 4.0, 7.9, 13.2±0.2 degrees/2 theta [Cu Kα radiation (λ=1.5406 Å)], by dissolving the crystal form A of the compound of formula (I) in acetone.

In an additional aspect, the invention refers to the crystal Form 2 of the compound of formula (I), obtained (or obtainable) by dissolving the crystal Form A of the compound of formula (I) in acetone.

In another aspect, the invention provides the crystal Form 2 of the compound of formula (I), for use as a medicament.

In a further aspect, the invention provides a pharmaceutical composition comprising the crystal Form 2 of the compound of formula (I) and one or more pharmaceutically acceptable carriers and/or excipients for use as a medicament.

In a still further aspect, the invention provides the use of the crystal Form 2 of the compound of formula (I) as defined above for the manufacture of a medicament for the prevention and/or treatment of an inflammatory or obstructive respiratory disease.

In a further aspect, the invention provides a pharmaceutical composition comprising the crystal Form 2 of the compound of formula (I) as defined above and one or more pharmaceutically acceptable carriers and/or excipients for use in the prevention and/or treatment of an inflammatory or obstructive respiratory disease.

In a further aspect, the invention provides the use of a pharmaceutical composition comprising the crystal Form 2 of the compound of formula (I) as defined above and one or more pharmaceutically acceptable carriers and/or excipients for the manufacture of a medicament for the prevention and/or treatment of an inflammatory or obstructive respiratory disease.

In another aspect, the present invention provides a method for preventing and/or treating an inflammatory or obstructive respiratory disease in human, the method comprising administering an effective amount of the crystal Form 2 of the compound of formula (I) as defined above.

In a further aspect, the present invention provides a method for preventing and/or treating an inflammatory or obstructive respiratory disease in human, the method comprising administering an effective amount of pharmaceutical composition comprising the crystal Form 2 of the compound of formula (I) as defined above and one or more pharmaceutically acceptable carriers and/or excipients.

In a further aspect, the invention refers to a device comprising a pharmaceutical composition comprising the crystal Form 2 of the compound of formula (I).

### BRIEF DESCRIPTION OF THE FIGURES

FIG. 1: XRPD of the crystal Form 2 of the compound of formula (I)

FIG. 2: XRPD of the crystal Form A of the compound of formula (I)

FIG. 3: DSC of the crystal Form 2 of the compound of formula (I)

FIG. 4: DSC of the crystal Form A of the compound of formula (I)

FIG. 5: TGA of the crystal Form 2 of the compound of formula (I)

FIG. 6: TGA of the crystal Form A of the compound of formula (I)

### DETAILED DESCRIPTION OF THE INVENTION

### Definitions

Unless defined otherwise, all technical and scientific terms used herein have the same meanings as commonly understood by the skilled in the art.

The term “compound of the invention” refers to the crystal Form 2 of the compound of formula (I).

Unless otherwise specified, the compound of formula (I) of the present invention is intended to include also stereoisomers, tautomers or pharmaceutically acceptable salts or solvates thereof.

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “racemate” or “racemic mixture” refers to a composition composed of equimolar quantities of two enantiomeric species, wherein the composition is devoid of optical activity.

The term “tautomer” refers to each of two or more isomers of a compound that exist together in equilibrium and are readily interchanged by migration of an atom or group within the molecule.

The term “composition”, as in pharmaceutical composition, is intended to encompass a product comprising the active ingredient and any pharmaceutically acceptable excipient or carrier, as well as any product which results, directly or indirectly, from combination, complexation or aggregation of any two or more of the ingredients, or from dissociation of one or more of the ingredients, or from other types of reactions or interactions of one or more of the ingredients.

Accordingly, the pharmaceutical compositions of the invention comprehend any type of composition made by admixing the compound of the invention and pharmaceutically acceptable excipients and/or carriers.

The term “high level of chemical purity” refers to a crystal form wherein the total amount of readily detectable impurities as determined by standard methods of analysis, such as thin layer chromatography (TLC) or high performance liquid chromatography (HPLC) is less than 5%, advantageously less than 2.5%, even less than 1.0, or more preferably even less than 0.5% w/w.

The term “high level of crystallinity” refers to a crystal form wherein the percentage of crystallinity is equal to or higher than 90%, preferably higher than 95% w/w as determined by standard methods of analysis, such as X-ray powder diffraction or microcalorimetry.

The terms “polymorph”, “polymorphous form”, “crystal”, “crystal form”, “crystalline”, “crystalline form” and “form” mean a compound having a particular molecular packing arrangement in a crystal lattice. X-ray powder diffraction (XRPD), proton nuclear magnetic resonance (NMR), differential scanning calorimetry (DSC) and thermogravimetric analysis (TGA) are techniques that can be used for identifying and characterising properties of polymorphous forms, such as crystal Form 2 of the compound of formula (I) as described herein.

The term “XRPD” means X-ray powder diffraction. XRPD is a technique for providing an analytical characterisation of a sample. Polymorphous forms are characterised by having different XRPD patterns.

The term “NMR” means proton nuclear magnetic resonance. NMR spectra provide structural information about organic compounds based on energy absorption by hydrogen atoms.

The term “DSC” means differential scanning calorimetry. DSC is a thermoanalytical technique measuring the difference in the amount of heat required to increase the temperature of a test compound and a reference as a function of temperature. The output is a differential thermogram and can for instance be used to estimate melting temperatures of the test compound. It is used for characterising polymorphous forms of salts.

The term “TGA” means thermogravimetric analysis. TGA is a technique where the mass of a substance is monitored as a function of temperature as a sample is subjected to a controlled temperature program in a controlled atmosphere. It is used for characterising polymorphous forms of salts.

The term “room temperature”, abbreviated to RT, means a temperature in the range of about 15° C. to about 25° C., with an average of about 23° C.

The term “treating”, or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.

The term “preventing”, or “prevention” of a disease state includes causing the clinical symptoms of the disease state not to develop in a subject that may be exposed to or predisposed to the disease state, but does not yet experience or display symptoms of the disease state. For example, treating or preventing a respiratory disease or disorder includes treating or preventing the symptoms the disorder such as cough and/or urge to cough associated with a respiratory disease.

The present invention refers to the crystal form of the compound of formula (I), designated as crystal Form 2.

Surprisingly, the crystal Form 2 of the compound of formula (I), according to the invention, shows an increased solubility in Simulated Lung Fluid (SLF) with respect to the crystal Form A of the compound of formula (I), as known in the art. As it can be appreciated in Table 2 of Example 4 in the present experimental part, after 0.5, 1 and 3 hours, the crystal Form 2 of the compound of formula (I) shows an higher solubility in SLF of at least about 4.5-fold more than the crystal Form A of the compound of formula (I). The use of SLF, that mimics the physiologically lung fluid, is known to be a useful parameter to give a good understanding of the dissolution mechanism and possible in vivo behavior of a product, enhancing the predictive capability of the dissolution testing in terms of bioavailability and therapeutic effectiveness.

More advantageously, the crystal Form 2 of the compound of formula (I) according to the invention, unexpectedly shows a low risk of lung accumulation after repeated administration. As it can be appreciated in Table 3 of Example 5 in the present experimental part, the crystal Form 2 of the compound of formula (I) according to the invention shows a lung t_{1/2} of 7.7 hours, indicating a low risk of accumulation in the lung after repeated administration.

Even more advantageously, the lung t_{1/2} and lung MRTlast values, reported in Table 3 of Example 5 in the present experimental part, are predictive of the potential for crystal Form 2 of the compound of formula (I) of having a good in vivo bioavailability.

Additionally, the crystal Form 2 of the compound of formula (I) of the invention may also be useful in the treatment of a disease wherein the activity of PDE4 receptors is implicated and inhibition of PDE4 receptor activity is desired, or even a disease state which is mediated by PDE4 activity.

In one preferred embodiment, the present invention provides a crystal form of the compound of formula (I)

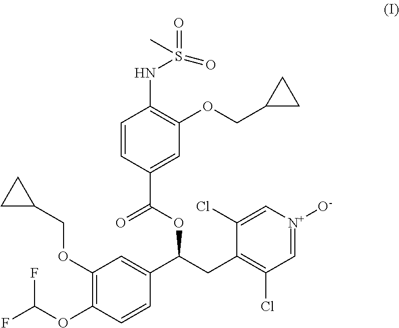 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]

designated as crystal Form 2, characterized by at least the following XRPD peaks: 4.0, 7.9, 13.2±0.2 degrees/2 theta [Cu Kα radiation (λ=1.5406 Å)].

In another preferred embodiment, the present invention provides a crystal form of the compound of formula (I) designated as crystal Form 2, wherein said crystal form is characterized by the following XRPD peaks: 4.0, 7.9, 13.2±0.2 degrees/2 theta [Cu Kα radiation (λ=1.5406 Å)].

In a further preferred embodiment, the present invention provides a crystal form of a compound of formula (I) designated as crystal Form 2, wherein said crystal form is characterized by the following XRPD peaks: 4.0, 7.9, 8.6, 13.2, 15.0±0.2 degrees/2 theta [Cu Kα radiation (λ=1.5406 Å)].

In a more preferred embodiment, the present invention provides a crystal form of a compound of formula (I) designated as crystal Form 2, wherein said crystal form is characterized by the following XRPD peaks: 4.0, 7.9, 8.6, 9.1, 12.2, 13.2, 15.0, 19.8, 20.9, 23.6±0.2 degrees/2 theta [Cu Kα radiation (λ=1.5406 Å)].

In another embodiment, the present invention also refers to the crystal Form 2 of the compound of formula (I), characterized by a DSC melting range of 120°-135° C. (heating rate 10° C./min, N₂ flow of 50 ml/min).

In one embodiment, the present invention also refers to a process for the preparation of the crystal Form 2 of the compound of formula (I), characterized by at least the following XRPD peaks 4.0, 7.9, 13.2±0.2 degrees/2 theta [Cu Kα radiation (λ=1.5406 Å)], comprising the step of dissolving the crystal form A of the compound of formula (I) in acetone.

Thus, the invention also refers to the crystal Form 2 of the compound of formula (I), thus obtained, according to the embodiments herein below.

In another embodiment, the present invention refers to a process for the preparation of the crystal Form 2 of the compound of formula (I), characterized by at least the following XRPD peaks 4.0, 7.9, 13.2±0.2 degrees/2 theta [Cu Kα radiation (λ=1.5406 Å)], comprising the steps of dissolving the crystal Form A of the compound of formula (I) in acetone, stirring and isolating the crystal solid thus formed.

In another preferred embodiment, the present invention refers to a process as defined above for the preparation of the crystal Form 2 of the compound of formula (I), characterized by at least the following XRPD peaks 4.0, 7.9, 13.2±0.2 degrees/2 theta [Cu Kα radiation (λ=1.5406 Å)], wherein the stirring is carried out at a temperature comprised between 15° C. and 30° C.

In another preferred embodiment, the present invention refers to a process as defined above for the preparation of the crystal Form 2 of the compound of formula (I), characterized by at least the following XRPD peaks 4.0, 7.9, 13.2±0.2 degrees/2 theta [Cu Kα radiation (λ=1.5406 Å)], wherein the crystal solid is isolated by microfiltration.

In another preferred embodiment, the present invention refers to a process as defined above for the preparation of the crystal Form 2 of the compound of formula (I), characterized by at least the following XRPD peaks 4.0, 7.9, 13.2±0.2 degrees/2 theta [Cu Kα radiation (λ=1.5406 Å)], wherein the stirring is carried out at a temperature comprised between 15° C. and 30° C. and the crystal solid is isolated by microfiltration.

In a further embodiment, the present invention also refers to a process for the preparation of the crystal Form 2 of the compound of formula (I), characterized by the following XRPD peaks 4.0, 7.9, 8.6, 13.2, 15.0±0.2 degrees/2 theta [Cu Kα radiation (λ=1.5406 Å)], comprising the step of dissolving the crystal form A of the compound of formula (I) in acetone.

In another embodiment, the present invention refers to a process for the preparation of the crystal Form 2 of the compound of formula (I), characterized by the following XRPD peaks 4.0, 7.9, 8.6, 13.2, 15.0±0.2 degrees/2 theta [Cu Kα radiation (λ=1.5406 Å)], comprising the steps of dissolving the crystal Form A of the compound of formula (I) in acetone, stirring and isolating the crystal solid thus formed.

In another preferred embodiment, the present invention refers to a process for the preparation of the crystal Form 2 of the compound of formula (I), characterized by the following XRPD peaks 4.0, 7.9, 8.6, 13.2, 15.0±0.2 degrees/2 theta [Cu Kα radiation (λ=1.5406 Å)], wherein the stirring is carried out at a temperature comprised between 15° C. and 30° C.

In a further preferred embodiment, the present invention refers to a process as defined above for the preparation of the crystal Form 2 of the compound of formula (I), characterized by the following XRPD peaks 4.0, 7.9, 8.6, 13.2, 15.0±0.2 degrees/2 theta [Cu Kα radiation (λ=1.5406 Å)], wherein the crystal solid is isolated by microfiltration.

In a further preferred embodiment, the present invention refers to a process as defined above for the preparation of the crystal Form 2 of the compound of formula (I), characterized by the following XRPD peaks 4.0, 7.9, 8.6, 13.2, 15.0±0.2 degrees/2 theta [Cu Kα radiation (λ=1.5406 Å)], wherein the stirring is carried out at a temperature comprised between 15° C. and 30° C., and the crystal solid is isolated by microfiltration.

In a further preferred embodiment, the present invention also refers to a process for the preparation of the crystal Form 2 of the compound of formula (I), characterized by the following XRPD peaks 4.0, 7.9, 8.6, 9.1, 12.2, 13.2, 15.0, 19.8, 20.9, 23.6±0.2 degrees/2 theta [Cu Kα radiation (λ=1.5406 Å)], comprising the step of dissolving the crystal form A of the compound of formula (I) in acetone.

In another embodiment, the present invention refers to a process for the preparation of the crystal Form 2 of the compound of formula (I), characterized by the following XRPD peaks 4.0, 7.9, 8.6,9.1, 12.2, 13.2, 15.0, 19.8, 20.9, 23.6±0.2 degrees/2 theta [Cu Kα radiation (λ=1.5406 Å)], comprising the steps of dissolving the crystal Form A of the compound of formula (I) in acetone, stirring and isolating the crystal solid thus formed.

In another preferred embodiment, the present invention refers to a process as defined above for the preparation of the crystal Form 2 of the compound of formula (I), characterized by the following XRPD peaks 4.0, 7.9, 8.6,9.1, 12.2, 13.2, 15.0, 19.8, 20.9, 23.6±0.2 degrees/2 theta [Cu Kα radiation (λ=1.5406 Å)], wherein the stirring is carried out at a temperature comprised between 15° C. and 30° C.

In a further preferred embodiment, the present invention refers to a process as defined above for the preparation of the crystal Form 2 of the compound of formula (I), characterized by the following XRPD peaks 4.0, 7.9, 8.6,9.1, 12.2, 13.2, 15.0, 19.8, 20.9, 23.6±0.2 degrees/2 theta [Cu Kα radiation (λ=1.5406 Å)], and the crystal solid is isolated by microfiltration.

In even further preferred embodiment, the present invention refers to a process as defined above for the preparation of the crystal Form 2 of the compound of formula (I), characterized by the following XRPD peaks 4.0, 7.9, 8.6,9.1, 12.2, 13.2, 15.0, 19.8, 20.9, 23.6±0.2 degrees/2 theta [Cu Kα radiation (λ=1.5406 Å)], wherein the stirring is carried out at a temperature comprised between 15° C. and 30° C., and the crystal solid is isolated by microfiltration.

In an additional aspect, the present invention provides a pharmaceutical composition for inhalation comprising the crystal Form 2 of the compound of formula (I), in combination with suitable carriers and/or excipients.

In another aspect, the present invention provides the crystal Form 2 of the compound of formula (I), for use in the prevention and/or treatment of an inflammatory or obstructive respiratory disease.

In a preferred embodiment, the present invention provides the crystal Form 2 as defined above, for use in the prevention and/or treatment of an inflammatory or obstructive respiratory disease, wherein the inflammatory or obstructive respiratory diseases are selected from: asthma, chronic obstructive pulmonary disease (COPD), bronchiectasis, chronic bronchitis, lung fibrosis, idiopathic pulmonary fibrosis, cystic fibrosis, pneumonia, acute respiratory distress syndrome (ARDS), pulmonary emphysema, smoking-induced emphysema and cystic fibrosis.

The present invention is also directed to a pharmaceutical composition comprising the crystal Form 2 of the compound of formula (I) and one or more pharmaceutically acceptable carriers and/or excipients.

Suitable excipients can be selected among those in the art, and they can include carriers, diluents, wetting agents, emulsifying agents, binders, coatings, fillers, glidants, lubricants, disintegrants, preservatives, surfactants, pH buffering substances and the like. Examples of excipients and their use are provided in the Handbook of Pharmaceutical Excipients, 5th ed. (2006), Ed. Rowe et al., Pharmaceutical Press.

The most suitable dosage level may be determined by any known suitable method. It will be understood, however, that the specific amount for any particular patient will depend upon a variety of factors, including, for example, the activity of the crystal Form 2 of the compound of formula (I), the age, body weight, diet, general health and sex of the patient, time of administration, the route of administration, the rate of excretion, the use of any other drugs, and the severity of the disease to be treated.

In one embodiment, the crystal Form 2 of the compound of formula (I) is preferably in the form of microparticles, even more preferably for administration by inhalation. Said microparticle may be prepared by a variety of techniques known in the art, including spray-drying, freeze-drying and micronisation.

In one embodiment, a composition of the invention is prepared in form of a suspension, suitable for delivery by a nebuliser or as an aerosol in a liquid propellant, even more preferably for use in a pressurised metered dose inhaler (pMDI). Suitable propellants for use in a pMDI are known to the skilled person, and include HFA-227, preferably HFA-134a and more preferably HFA152a.

In a preferred embodiment, a composition of the invention is in the form of a dry powder, more preferably for the use in a dry powder inhaler (DPI).

Microparticles for delivery by administration may be formulated with excipients that aid delivery and release. For example, in a dry powder formulation, microparticles may be formulated with large carrier particles that aid flow from the DPI into the lung. Suitable carrier particles are known in the art and include e.g. lactose particles.

The agents of the invention may be administered in inhaled form. Aerosol generation can be carried out using, for example, pressure-driven jet atomizers or ultrasonic atomizers, preferably using propellant-driven metered aerosols or propellant-free administration of micronised crystal Form 2 of the compound of formula (I) from, for example, inhalation capsules or other “dry powder” delivery systems.

In a preferred embodiment, the invention refers to the crystal Form 2 of the compound of formula (I) in the form of capsules.

As above described, the present invention is directed to the crystal Form 2 of the compound of formula (I) for use as a medicament.

According to a preferred embodiment, the present invention refers to the use of the crystal Form 2 of the compound of formula (I) for the preparation of a medicament for the treatment of an inflammatory or obstructive pulmonary disease, preferably the disease is selected from: asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

The present invention is also directed to a pharmaceutical composition comprising the crystal Form 2 of the compound of formula (I) and one or more pharmaceutically acceptable carriers and/or excipients for use as a medicament.

In a preferred embodiment, the present invention is directed to the crystal Form 2 of the compound of formula (I) for use for the prevention and/or treatment of an inflammatory or obstructive respiratory disease.

In another preferred embodiment, the present invention is directed to a pharmaceutical composition comprising the crystal Form 2 of the compound of formula (I) and one or more pharmaceutically acceptable carriers and/or excipients, for use for the prevention and/or treatment of an inflammatory or obstructive respiratory disease.

In a further preferred embodiment, the present invention provides a method for preventing and/or treating an inflammatory or obstructive respiratory disease, the method comprising administering an effective amount of the crystal Form 2 of the compound of formula (I).

In another preferred embodiment, the present invention provides a method for preventing and/or treating an inflammatory or obstructive respiratory disease, the method comprising administering an effective amount of pharmaceutical composition comprising the crystal Form 2 of the compound of formula (I) and one or more pharmaceutically acceptable carriers and/or excipients.

In a still further preferred embodiment, the inflammatory or obstructive respiratory diseases mentioned above are selected from asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

Any suitable route of administration may be employed for providing a mammal, especially a human, with an effective dosage of the crystal Form 2 of the compound of formula (I).

The magnitude of prophylactic or therapeutic dose of the crystal Form 2 of the compound of formula (I) will, of course, vary with the nature of the severity of the condition to be treated and with its route of administration, and will generally be determined by clinical trial as required in the pharmaceutical art.

It will also vary according to the age, weight and response of the individual patient.

In therapeutic use, the crystal Form 2 of the compound of formula (I) may be administered by any convenient, suitable or effective route.

Suitable routes of administration are known, and include oral, intravenous, rectal, parenteral, topical, ocular, nasal, buccal and pulmonary (by inhalation).

The crystal Form 2 of the compound of formula (I) may be dosed as described depending on the inhaler system used. In addition to the active compound, the administration forms may additionally contain excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

The invention is also directed to a device comprising a pharmaceutical composition comprising the crystal Form 2 of the compound of formula (I) as described above according to the invention, in form of a single-or multi-dose dry powder inhaler, capsules or a metered dose inhaler (pMDI).

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321).

In a more preferred embodiment, the invention provides a process for the preparation of the crystal Form 2 of the compound of formula (I), starting from crystal Form A of the compound of formula (I) obtained according to general synthetic route described in WO 2015/059050.

The following non-limiting examples are illustrative for the disclosure and are not to be construed as to be in any way limiting for the scope of the invention.

### EXPERIMENTAL PART

### Instruments

### X-Ray Powder Diffraction (XRPD)

The crystalline state of the Form 2 of the compound of formula (I) samples was investigated by X-ray powder diffraction (Empyrean V2.0, Panalytical) equipped with Cu radiation source (Cu Kα λ=1.5406 Å). Samples were placed on Si zero background sample holders spinning with revolution time 4 s. The measurements were performed in reflection mode, 2 theta scan from 1.5 to 45°, step size 0.02°, soller slit 0.02 rad, divergence slit ⅛°, antiscatter slit ¼°. The variable temperature and humidity XRPD analysis were carried out with Anton Paar CHC+ camera equipped with CCU100 temperature control and MHG-32 humidity generator. The measurements were performed in reflection mode, 2 theta scan from 1.5 to 45°, step size 0.02°, soller slit 0.02 rad, divergence slit ⅛°, antiscatter slit ¼°.

### Thermogravimetric Analysis (TGA)

TGA analysis was performed using a TA Instruments thermogravimetric analyzer Discovery equipped with a computer analyzing system (TRIOS). Each sample (5-10 mg) was placed in an aluminum sample pan, inserted into the TGA furnace, and accurately weighed. The furnace was first equilibrated at 25° C., and then heated under nitrogen (flow rate 30 mL/min) at a rate of 10° C./min, up to a final temperature of 230° C. Nickel was used as the calibration standard.

### Differential Scanning Calorimetry (DSC)

DSC analysis was performed using a TA Instruments differential scanning calorimeter Discovery equipped with a computer analyzing system (TRIOS).

About 1-5 mg of each sample were placed into a Perkin Elmer Aluminum DSC pan. The pan was covered with a lid, without crimping it. The sample cell was equilibrated at 0° C. and heated under a nitrogen purge (50 mL/min). All samples were given similar thermal histories by linearly heating to 300° C. at a heating rate of 10° C./min. Indium metal was used as the calibration standard. Modulated DSC (mDSC) analysis was performed using the same TA Instruments Discovery DSC. All the samples were analyzed with a period of 60 s and an amplitude of 1° C.

### Nuclear Magnetic Resonance Spectroscopy (1H NMR)

The 1H NMR spectra was performed on a Bruker AVANCE III HD 600 spectrometer operating at 600 MHz (proton frequency). The spectrometer was equipped with a 5 mm TCI inverse triple resonance cryoprobe H-C/N-D-0.5-Z ATMA. The probe is fitted with an actively shielded single axis Z-gradient and allowed simultaneous decoupling on multiple X-nuclei such as 13C and 15N as well as automatic tuning and matching.

### Ultra Performance Liquid Chromatography Coupled and Mass Spectrometry (UPLC/MS)

### LC Conditions

UPLC instrument: Waters Acquity
Column: Acquity UPLC CSH C18 1.7 um 50×2.10 (Waters)
Column Temperature (° C.): 55
Mobile phases: HCOONH₄ 0.025M pH3 (A); MeCN+0.1% HCOOH (B)
Flow (mL/min): 0.5 (split in MS 1:10)
Stop Time (mins): 10
Gradient:

| Time (min) | % A | % B |
|---|---|---|
| 0.00 | 90.0 | 10.0 |
| 0.50 | 90.0 | 10.0 |
| 3.00 | 75.0 | 25.0 |
| 7.50 | 50.0 | 50.0 |
| 8.00 | 20.0 | 80.0 |
| 8.10 | 90.0 | 10.0 |
| 10.00 | 90.0 | 10.0 |

Injection Volume (μL): 2
Sample Solvent: Acetonitrile/Water 60/40

### MS Conditions

MS instrument: Waters XEVO-TQS
Acquisiton Mode: Multiple Reaction Monitoring (MRM) of 2 mass pairs
Parent (m/z): 687.15; daughter (m/z): 402.08, 456.12
Ionization mode: ES+
Capillary (kV): 1.60
Cone (V): 20.0
Source Offset (V): 60.0
Source Temperature (° C.): 150
Desolvation Temperature (° C.): 250
Cone Gas Flow (L/Hr): 250
Desolvation Gas Flow (L/Hr): 500
Total run time (min): 5

### Crystallization Systems

Polar Bear Plus, Cambridge Reactor Design was used for slurry at 50° C. and temperature cycling experiments while for crystallization optimization Crystal16 (Technobis Crystallization systems) was used.

### EXAMPLES

### Example 1: Preparation of the Crystal Form 2 of the Compound of Formula (I)

The crystal Form 2 of the compound of formula (I) was prepared starting from the crystal Form A of the compound of formula (I), prepared according to the procedure disclosed in Example 10 of WO 2015/059050 A1, according to the following preparation.

2 g of the crystal Form A of the compound of formula (I) was dissolved in 200 ml of acetone. The resulting organic phase was stirred at RT for 2 h, microfiltered and dried overnight in open vial at room temperature affording a white solid (99 mg, quantitative yield).

### Example 2: Characterisation of the Crystal Form 2 of the Compound of Formula (I)

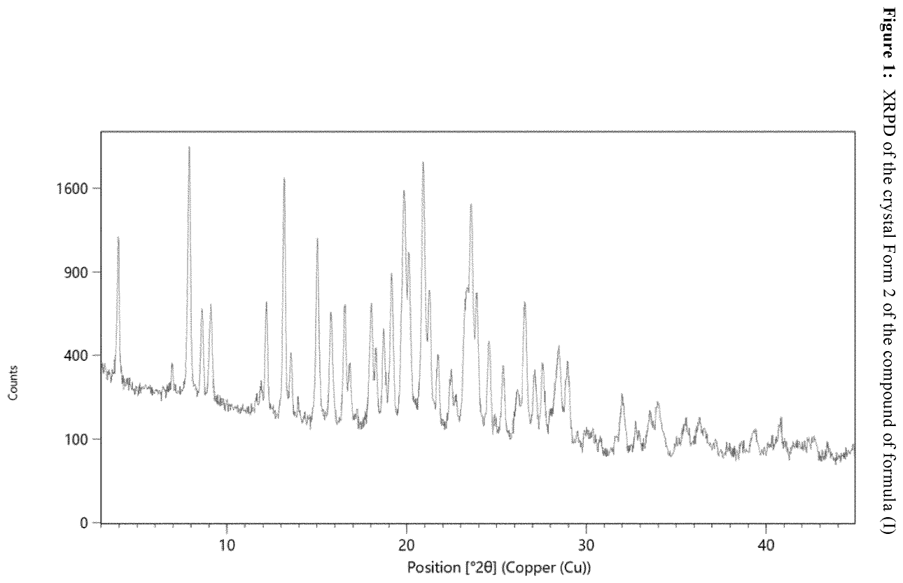

The XRPD pattern of the crystal Form 2 of the compound of formula (I) obtained according to preparation of Example 1 is shown in FIG. 1. The following characteristic diffraction peaks at diffraction angles 2-theta were identified for the crystal Form 2 of the compound of formula (I): 4.0, 7.9, 8.6, 9.1, 12.2, 13.2, 15.0, 19.8, 20.9 and 23.6±0.2 degrees/2 theta [Cu Kα radiation (λ=1.5406 Å)].

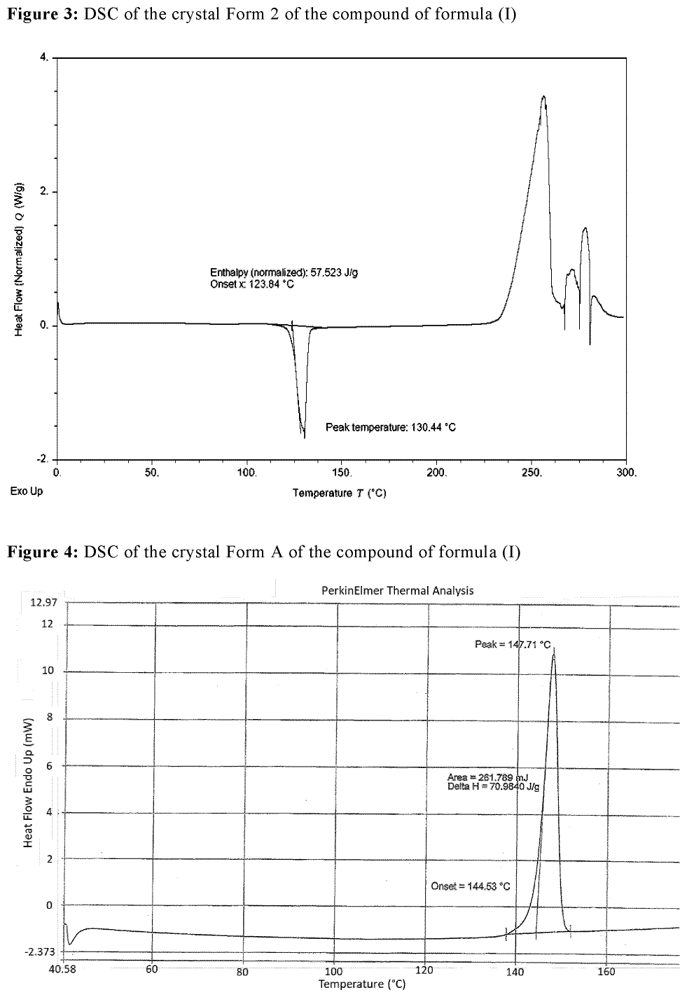

A DSC profile of the crystal Form 2 of the compound of formula (I) is shown in FIG. 3. No thermal event is shown until a first endothermic peak with an onset temperature of about 123.84° C. and a peak temperature of about 130.44° C., which is due to the melting of the sample.

The crystal Form 2 of the compound of formula (I) is characterized by a DSC melting range of 120°-135° C. (heating rate 10° C./min, N₂ flow of 50 ml/min).

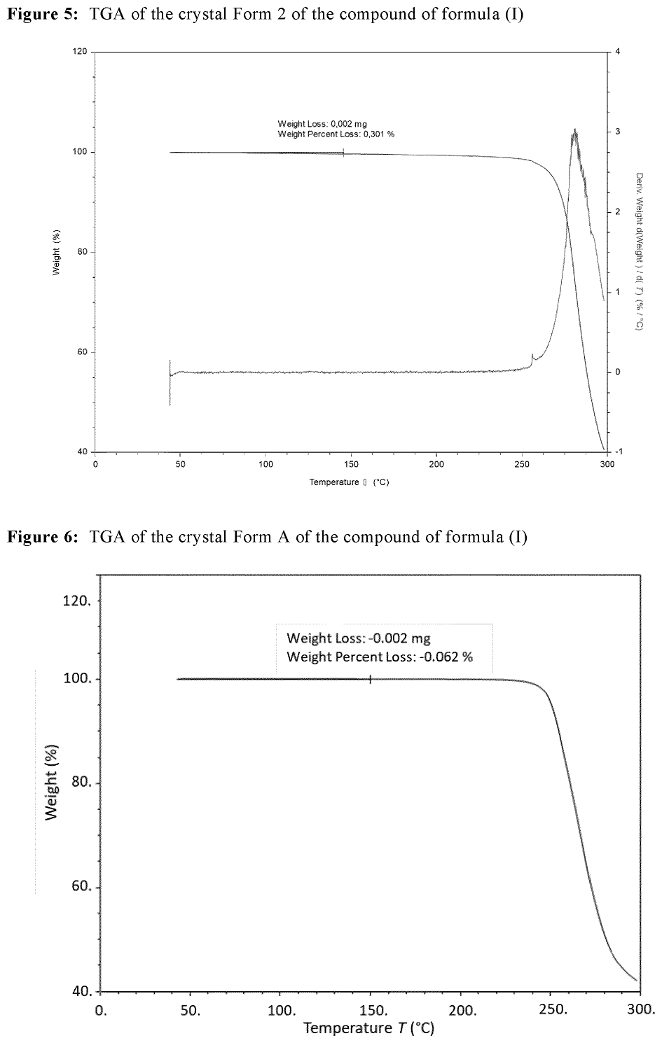

A TGA profile of the crystal Form 2 of the compound of formula (I) is shown in FIG. 5. Negligible weight loss was observed until around 250° C. The TGA profile is indicative of the anhydrous nature of the crystal Form 2 of the compound of formula (I).

The proton NMR (1H-NMR, δ ppm, DMSO-d6) of the crystal Form 2 of the compound of formula (I) shows the following signals: 0.38 (dq, 4H, CH₂), 0.58 (dq, 4H, CH₂), 1.21 (m, 1H, CH), 1.30 (m, 1H, CH), 3.12 (s, 3H, CH₃), 3.35 (m, 2H, CH₂), 3.61 (dd, 1H, CH), 3.94 (m, 4H, CH₂), 6.18 (dd, 1H, CH), 7.07 (m, 1H, CHₐᵣₒₘ), 7.20 (m, 2H, CHₐᵣₒₘ), 7.41 (d, 1H, CHₐᵣₒₘ), 7.50 (d, 1H, CHₐᵣₒₘ), 7.60 (dd, 1H, CHₐᵣₒₘ), 8.57 (s, 2H, CHₐᵣₒₘ), 9.19 (s_{br}, 1H, NH).

### Example 3: Characterisation of the Crystal Form A of the Compound of Formula (I)

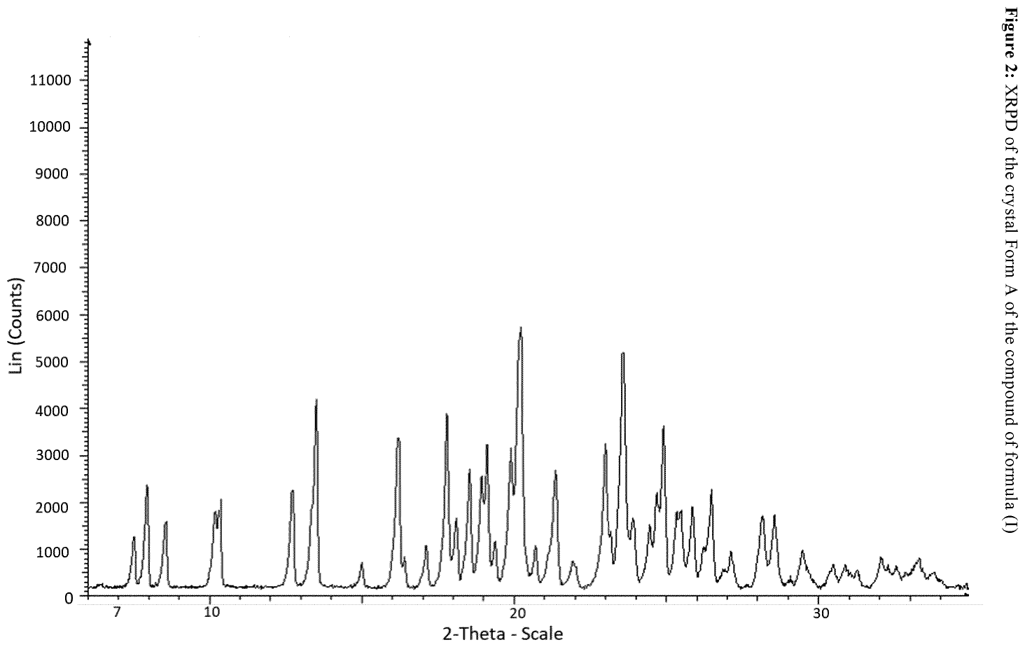

The XRPD pattern of the crystal Form A of the compound of formula (I) according to Example 10 and FIG. 4 of WO 2015/059050 A1, is shown in FIG. 2.

A DSC profile of the crystal Form A of the compound of formula (I) is shown in FIG. 4, according to Example 10 and FIG. 6 of WO 2015/059050 A1. The crystal Form A of the compound of formula (I) is characterized by a melting range of 144°-147° C., determined by DSC at a scan rate of 10° C./min.

A TGA profile of the crystal Form A of the compound of formula (I) is shown in FIG. 6.

The proton NMR (1H-NMR, δ ppm, DMSO-d6) of the crystal Form A of the compound of formula (I) shows the following signals: 0.30 (dq, 4H, CH₂), 0.55 (dq, 4H, CH₂), 1.12 (m, 1H, CH), 1.31 (m, 1H, CH), 3.30 (s, 3H, CH₃), 3.34 (m, 2H, CH₂), 3.59 (dd, 1H, CH), 3.90 (m, 4H, CH₂), 6.14 (dd, 1H, CH), 7.03 (m, 1H, CHₐᵣₒₘ), 7.20 (m, 2H, CHₐᵣₒₘ), 7.38 (d, 1H, CHₐᵣₒₘ), 7.48 (d, 1H, CHₐᵣₒₘ), 7.59 (dd, 1H, CHₐᵣₒₘ), 8.56 (s, 2H, CHₐᵣₒₘ), 9.18 (s_{br}, 1H, NH).

### Example 4: Solubility of the Crystal Form A and Crystal Form 2 of the Compound of Formula (I) in Simulated Lung Fluid (SLF)

### Preparation of Simulated Lung Fluid

A study of solubility in SLF for the characterization of the crystal Form 2 and crystal Form A of the compound of formula (I), was performed. The use of SLF, that mimics the physiologically lung fluid, can give a good understanding of the dissolution mechanism and possible in vivo behavior of a product, enhancing the predictive capability of the dissolution testing in terms of bioavailability and therapeutic effectiveness.

The experimental method used is based on saturation shake-flask method. The samples were prepared by adding, respectively, an excess of the crystal Form 2 of the compound of formula (I) and an excess of the crystal Form A of the compound of formula (I), to the solubility medium SLF. The solubility concentration value is the thermodynamic equilibrium concentration, determined by assaying the solute concentration of the filtrate from a saturated solution, using an analytical method by UPLC-MS (MRM quantification; calibration: from 0.01 to 10 μg/ml; every batch have been tested in duplicate and samples have been filtered and diluted 1/10 in duplicate).

The SLF used for the tests were prepared analogously to literature (Simulated Biological Fluids with Possible Application in Dissolution Testing, Dissolution Technologies, 2011, dx.doi.org/10.14227/DT180311P15, table 11, page 2215). To mimic better the lung fluid, a surfactant, particularly Tween 80, has been added (0.02% v/v).

TABLE 1
SLF composition used for solubility testing of crystal Form
A and crystal Form 2 of the compound of formula (I)

| Component | Amount (g) in 1 L H₂O |
|---|---|
| MgCl₂•6 H₂O | 0.2033 |
| NaCl | 6.019 |
| KCl | 0.2982 |
| Na₂HPO₄•7 H₂O | 0.268 |
| Na₂SO₄ | 0.071 |
| CaCl₂•2 H₂O | 0.3676 |
| NaH₃C₂O₂•3 H₂O | 0.9526 |
| NaHCO₃ | 2.6043 |
| Na₃H₅C₆O₇•2 H₂O | 0.097 |

The solubility profile of the crystal Form 2 and the crystal Form A of the compound of formula (I) was determined in SLF adjusted with acetic acid (1M) to pH of 6.5, in order to simulate an inflammation condition in the lung. The experiment was conducted at 37° C.

The following experimental conditions were applied for the SLF dissolution profile study respectively for the crystal Form 2 and the crystal Form A of the compound of formula (I). About 2.5 mg of the crystal Form 2 or the crystal Form A of the compound of formula (I) was dispersed in 2.5 ml of SLF with stirring system. The concentration of material in solution was determined after 0.5, 1 and 3 hours. In each time interval, the vessel stirring was stopped, and 180-250 μl of each samples have been collected and filtered by RC Membrane Syringe Filters 0.45 μm and injected in UPLC apparatus.

The results of the solubility tests of the crystal Form A and the crystal Form 2 of the compound of formula (I) are summarized in the following Table 2.

TABLE 2
Solubility of the crystal Form A and crystal
Form 2 of the compound of formula (I)

| Time (h) | Crystal Form A (μg/ml) | Crystal Form 2 (μg/ml) |
|---|---|---|
| 0.5 | 0.16 | 0.74 |
| 1 | 0.18 | 0.89 |
| 3 | 0.20 | 0.88 |

Table 2 clearly shows a higher solubility, of at least about 4.5-fold more, of the crystal Form 2 of the compound of formula (I) with respect to the crystal Form A of the compound of formula (I) in SLF at 0.5, 1 and 3 hours.

### Example 5: Lung t_{1/2} and Lung MRTₗₐₛₜ of the Crystal Form 2 of the Compound of Formula (I)

Crystal Form 2 of the compound of formula (I) was intratracheally administered to Sprague-Dawley rats using the PreciseInhale system at the dose of 10 μg/lung deposited dose. Powder aerosols was generated using 40 bar generating pressure. A total number of 24 Sprague-Dawley male rats was used for PK investigation; 8 time points for each study, n=3 rats/time point. Before administration, rats were anesthetized with sevoflurane and endotracheally intubated using appropriate steel catheters. Aerosol administration was performed connecting the catheter to the PreciseInhale system, previously set to deliver 10 μg/lung deposited dose to each animal.

TABLE 3
Lung t_{1/2} and Lung MRTₗₐₛₜ of the crystal
Form 2 of the compound of formula (I)

| Lung t_{1/2} (h) | Lung MRTₗₐₛₜ (h) |
|---|---|
| 7.7 | 9.4 |

Table 3 shows the Lung t_{1/2} and Lung MRTₗₐₛₜ of the crystal Form 2 of the compound of formula (I) after intratracheal administration to Sprague-Dawley rats at the dose of 10 μg/lung (deposited dose).

Lung t_{1/2} is the terminal half-life, calculated by the formula ln(2)/λz, where λz is the first-order rate constant associated with the terminal (log-linear) portion of the curve. Estimated by linear regression of time vs. log concentration; i.e. the time it takes for the concentration of the drug in the lung to be reduced by 50%.

Lung MRTₗₐₛₜ is the mean residence time from the time of dosing to the time of the last measurable concentration; i.e. the average time a molecule stays in the lung.

The lung t_{1/2} value of the crystal Form 2 of the compound of formula (I) corresponds to a short lung retention, indicating a potential low risk of lung accumulation after repeated administration.

The lung t_{1/2} and lung MRTₗₐₛₜ values are indicative of the potential for the crystal Form 2 of the compound of formula (I) of having a good in vivo bioavailability.

1: A crystal form of the compound of formula (I) 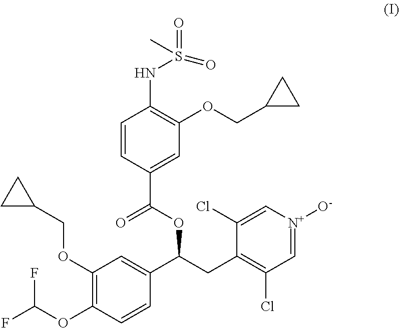 [SMILES: CS(=O)(=O)Nc1ccc(C(=O)O[C@@H](Cc2c(Cl)c[n+]([O-])cc2Cl)c2ccc(OC(F)F)c(OCC3CC3)c2)cc1OCC1CC1]
designated as crystal Form 2, characterized by at least the following XRPD peaks: 4.0, 7.9, 13.2±0.2 degrees/2 theta [Cu Kα radiation (α=1.5406 Å)].

2: A crystal Form 2 according to claim 1, characterized by the following XRPD peaks: 4.0, 7.9, 8.6, 13.2, 15.0±0.2 degrees/2 theta [Cu Kα radiation (λ=1.5406 Å)].

3: A crystal Form 2 according to claim 1, characterized by the following XRPD peaks: 4.0, 7.9, 8.6,9.1, 12.2, 13.2, 15.0, 19.8, 20.9, 23.6±0.2 degrees/2 theta [Cu Kα radiation (λ=1.5406 Å)].

4: A crystal Form 2 according to claim 1, characterized by a DSC melting range of 120°-135° C. (heating rate 10° C./min, N₂ flow of 50 ml/min).

5: A process for the preparation of the crystal Form 2 of the compound of formula (I), according to claim, comprising dissolving the crystal Form A of the compound of formula (I) in acetone.

6: A process according to claim 5, further comprising stirring and isolating the crystal Form 2 thus formed.

7: A process according to claim 6, wherein the stirring is carried out at a temperature of 15° C. to 30° C.

8: A process according to claim 1, wherein the crystal Form 2 is isolated by microfiltration.

9: A pharmaceutical composition comprising the crystal Form 2 of the compound of formula (I) as defined in claim 1 and one or more pharmaceutically acceptable carriers and/or excipients.

10: A pharmaceutical composition according to claim 9, formulated in form of a dry powder.

11. (canceled)

12: A method for the prevention and/or treatment of an inflammatory or obstructive respiratory disease, comprising administering to a subject in need thereof an effective amount of the crystal Form 2 according to claim 1.

13: The method crystal according to claim 12, wherein the inflammatory or obstructive respiratory disease is selected from asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

14: A method for the prevention and/or treatment of an inflammatory or obstructive respiratory disease, comprising administering to a subject in need thereof an effective amount of the pharmaceutical composition according to claim 9.

15: The method according to claim 14, wherein the inflammatory or obstructive respiratory disease is selected from: asthma, chronic obstructive pulmonary disease (COPD), bronchiectasis, chronic bronchitis, lung fibrosis, idiopathic pulmonary fibrosis, pneumonia, acute respiratory distress syndrome (ARDS), pulmonary emphysema, smoking-induced emphysema and cystic fibrosis.

16: A device comprising a pharmaceutical composition comprising the crystal Form 2 of the compound of formula (I), as defined in claim 1.

17: The device according to claim 16 which is in a form of a single-or multi-dose dry powder inhaler or a capsule.

18: The device according to claim 16 which is in a form of a pressurised metered dose inhaler (pMDI).

19: The crystal Form 2 of the compound of formula (I), as defined in claim 1, which obtained by dissolving the crystal Form A of the compound of formula (I) in acetone.
